(19) United States
(12) Patent Application Publication
(10) Pub. No.: US 2023/0105650 A1
(43) Pub. Date: Apr. 6, 2023

(54) Analytical Assessment Of Bacterial Endotoxin

(71) Applicant: DePuy Synthes Products, Inc., Raynham, MA (US)

(72) Inventors: Allan Kimble, Downingtown, PA (US); Gerald McDonnell, Basking Ridge, NJ (US); James Hauschild, Cranbury, NJ (US)

(21) Appl. No.: 17/930,491

(22) Filed: Sep. 8, 2022

Related U.S. Application Data
(60) Provisional application No. 63/242,195, filed on Sep. 9, 2021.

(51) Int. Cl. G01N 33/50; G01N 21/33

(52) U.S. Cl. CPC G01N 33/5014; G01N 21/33

(57) ABSTRACT

Provided herein are methods for spectrophotometric detection of bacterial endotoxin in a fluid sample, wherein such detection can occur at low concentrations of contamination, and uses systems that permit offline or inline and optionally continuous assessment. Also disclosed are methods for identifying a particular bacterial source of an endotoxin in a fluid sample.

### CROSS-REFERENCE TO RELATED APPLICATIONS

The present application claims the benefit of priority to U.S. Provisional Application No. 63/242,195, filed Sep. 9, 2021, the entire contents of which are incorporated herein by reference.

### TECHNICAL FIELD

The present disclosure relates to detection of bacterial endotoxin.

### BACKGROUND

Bacterial endotoxin is associated with the external cell walls of Gram negative bacteria. It elicits a toxic response when it is released from the cell wall and interacts with an organism. Chemically, BET is a lipopolysaccharide (LPS). Testing of drugs and devices for pyrogenicity is required when their intended use would cause adverse patient responses if endotoxin were present above levels set by regulations. In June 2012, the FDA published Guidance for Industry: Pyrogen and Endotoxins Testing: Questions and Answers. This guidance cites AAMI ST72 “Bacterial endotoxins—Test methods, routine monitoring, and alternatives to batch testing” as well as USP <161>Medical Devices—Bacterial Endotoxin and Pyrogen Tests, which describes the scope of the devices requiring this assay:
The methods and requirements in this chapter apply to assemblies or devices labeled sterile and nonpyrogenic that are in contact directly or indirectly with the cardiovascular system, lymphatic system, or cerebrospinal fluid.

The specific testing is described in USP <85> Bacterial Endotoxins Test that is harmonized with the corresponding texts of the European Pharmacopoeia and/or the Japanese Pharmacopoeia, thereby providing a global approach to BET testing. From this chapter comes the following:
The Bacterial Endotoxins Test (BET) is a test to detect or quantify endotoxins from Gram-negative bacteria using amoebocyte lysate from the horseshoe crab (Limulus polyphemus or Tachypleus tridentatus). There are three techniques for this test: the gel-clot technique, which is based on gel formation; the turbidimetric technique, based on the development of turbidity after cleavage of an endogenous substrate; and the chromogenic technique, based on the development of color after cleavage of a synthetic peptide-chromogen complex.
   Additional methodologies are in development or early commercial use (e.g., bacterial endotoxin testing using the recombinant Factor C (rFC) assay) and have only limited acceptance by the FDA. The rFC assay will still be considered an “Alternative Test”, subject to the validation requirements of USP <1225> or ICH Q2B6. Although regulatory authorities will accept the test results of the recombinant Factor C assay, a validation study must be performed for each product that will be tested using this method.

At present, the standard test for BET is the Limulus amebocyte lysate (LAL) assay. This reaction has been known since 1956 and the assay was described in the U.S. Federal Register in 1973 for the testing of drugs and devices. Concerns regarding the long-term viability of the practice of harvesting and using Limulus amebocyte lysate has given rise to the need for identifying alternative methods for quantification of BET, none of which have attained commercial realization or regulatory acceptance.

### SUMMARY

Disclosed herein are methods for detecting bacterial endotoxin in a fluid sample comprising exposing the fluid sample to ultraviolet radiation, spectroscopically sensing absorbance of the ultraviolet radiation by the sample at a wavelength in the range of 190-220 nm, and, based on the sensed absorption, determining whether the bacterial endotoxin is absent or present in the sample.

Also provided are methods for identifying a bacterial source of an endotoxin in a fluid sample comprising exposing the fluid sample to ultraviolet radiation, spectroscopically sensing the absorbance of the ultraviolet radiation by the sample at a wavelength in the range of 190-220 nm, increasing the concentration of the endotoxin in the fluid sample until an inflection in the absorbance spectrum sensed from the sample is observed, and, correlating the concentration of the endotoxin at which the inflection occurs to the bacterial source of the endotoxin.

The present disclosure also pertains to kits for detecting bacterial endotoxin in a fluid sample or determining a bacterial source of an endotoxin in a fluid sample comprising a spectrophotometer that is configured to scan the fluid sample at a wavelength in the range of 190-220 nm, a spectrophotometer cell having a radiation path length of about 8 to about 50 mm for containing the fluid sample during scanning by the spectrophotometer, and, a detergent that does not produce spectral interference in the spectral analytical range of 190-220 nm for adding to the fluid sample in a concentration of about 0.01-0.10% by volume.

### BRIEF DESCRIPTION OF THE DRAWINGS

FIG. 1A depicts an exemplary arrangement for using a real-time sensor for assessing the BET content of a source of fluid. FIG. 1B depicts details concerning the sensor apparatus itself.

FIG. 2 provides a generalized structure of bacterial endotoxin.

FIG. 3 depicts the spectral response obtained for different concentrations of Control Standard Endotoxin (CSE).

FIG. 4A provides a plot depicting CSE EU/mL versus absorbance, and FIG. 4B provides a logarithmic plot of the data (CSE Log EU/mL versus absorbance).

FIG. 5 depicts the effect of inflection of absorbance data as a result of aggregation of endotoxin molecules.

FIG. 6 provides respective absorbance spectra of 0.1% v/v aqueous solutions of three detergents.

FIG. 7 illustrates respective absorbance spectra for a blank sample and for each of three CSE samples at different concentrations to which SDS was added.

FIG. 8 depicts the spectral results of including 0.05% SDS to a sample containing endotoxin.

### DETAILED DESCRIPTION OF ILLUSTRATIVE EMBODIMENTS

The presently disclosed inventive subject matter may be understood more readily by reference to the following detailed description taken in connection with the accompanying examples, which form a part of this disclosure. It is to be understood that these inventions are not limited to the specific components, methods, or parameters described and/or shown herein, and that the terminology used herein is for the purpose of describing particular embodiments by way of example only and is not intended to be limiting of the claimed inventions.

The entire disclosures of each patent, patent application, and publication cited or described in this document are hereby incorporated herein by reference.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a detergent” is a reference to one or more of such detergents and equivalents thereof known to those skilled in the art, and so forth. Furthermore, when indicating that a certain element “may be” X, Y, or Z, it is not intended by such usage to exclude in all instances other choices for the element.

When values are expressed as approximations, by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. In some embodiments, “about X” (where X is a numerical value) refers to ±10% of the recited value, inclusive. For example, the phrase “about 8” can refer to a value of 7.2 to 8.8, inclusive. This value may include “exactly 8”. Where present, all ranges are inclusive and combinable. For example, when a range of “1 to 5” is recited, the recited range should be construed as optionally including ranges “1 to 4”, “1 to 3”, “1-2”, “1-2 & 4-5”, “1-3 & 5”, and the like. In addition, when a list of alternatives is positively provided, such a listing can also include embodiments where any of the alternatives may be excluded. For example, when a range of “1 to 5” is described, such a description can support situations whereby any of 1, 2, 3, 4, or 5 are excluded; thus, a recitation of “1 to 5” may support “1 and 3-5, but not 2”, or simply “wherein 2 is not included.”

As described above, there are few, if any, scientifically and commercially viable approaches for detecting bacterial endotoxin in fluid samples, such as within water storage facilities, that represent an alternative to the widely-used Limulus amebocyte lysate (LAL) assay. The present inventors have surprisingly discovered that BET can be spectroscopically detected using specific wavelengths of absorbance, and that such detection can occur at very low concentrations. This discovery enables online or inline measurements of BET with a sensitivity that is comparable to that of the LAL assay, but without the use of animal-derived reagents. The present inventors have also found that the non-linearity of absorbance of bacterial endotoxin based on the agglomeration behavior of these molecules in water can be used to identify the source of the endotoxin, e.g., the particular species that produced the detected endotoxin. Thus, using the presently disclosed methods and systems, it is possible not only to detect BET at commercially practical concentrations within a fluid, but also to pinpoint the bacterial species representing the source of the contamination.

Accordingly, provided herein are methods for detecting bacterial endotoxin in a fluid sample comprising exposing the fluid sample to ultraviolet radiation, spectroscopically sensing absorbance of the ultraviolet radiation by the sample at a wavelength in the range of 190-220 nm, and, based on the sensed absorption, determining whether the bacterial endotoxin is absent or present in the sample. The fluid sample may be water (e.g., representing a portion of a source of fluid held in a storage container as part of a water system for processing drinking water or water for industrial use, including food processing or preparation of commercial products or pharmaceuticals) or any other fluid (including water-containing fluids) in which bacterial endotoxins present a risk.

The present methods involve the use a spectrophotometer that is capable of measuring light intensity at a wavelength of 190-220 nm. In most instances, standard, commercially available spectrophotometers are appropriately configured. For example, the Cary 100 UV-VIS spectrophotometer from Agilent Technologies (Santa Clara, Calif.) can be used to measure absorbance at the specified wavelengths. In some embodiments the absorbance of UV radiation that is spectroscopically sensed is in a range of 195-220, 200-215, or 205-210 nm. For example, the wavelength of light that is sensed may be about 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, or 220 nm. As discussed more fully infra at Example 1, previous efforts to use spectroscopy for the detection of bacterial endotoxin identified an analytical range of 258-260 nm (see U.S. Pat. No. 4,195,225), but the present inventors used a series of Control Standard Endotoxin assays to determine that previous assessments of the absorption spectra for BET were not accurate. In contrast, the presently identified absorbance wavelengths enable accurate inline or online measurement of BET at low concentrations.

In some embodiments, the bacterial endotoxin is detected when present in the fluid sample in an amount of about 0.01 EU/mL to about 10 EU/mL. In certain embodiments, the bacterial endotoxin is detected when present in the fluid sample in an amount of about 0.1 EU/mL to about 10 EU/mL. For example, the bacterial endotoxin may be detected when present in the fluid sample in an amount of about 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, or 10 EU/mL.

The present methods may spectroscopically sense bacterial endotoxin while the sample is contained within a spectrophotometer cell having a relatively short radiation path length. For example, the path length of the spectrophotometer cell in accordance with the present methods may be about 8 to about 50 mm. In certain embodiments, the path length of the spectrophotometer cell in accordance with the present methods is about 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mm. As used herein, “spectrophotometer cell” can refer to (i) a cuvette or other vessel that can be used to house a fluid sample that is removed from a source of the fluid for testing using an external spectrophotometer device, or (ii) the pathway (e.g., tubing) through which a test fluid flows while being subjected to a real-time inline assessment of the BET content of a source of the fluid. In the case of an in-line system, the spectrophotometer cell may be removably immersed in a container that houses a fluid, such as a within a test vessel containing a quantity of fluid drawn from a main source of the fluid (e.g., a fluid storage vessel), or within a fluid storage vessel itself.

In some embodiments, the spectrophotometer may be operated in a continuous mode during the detection process, e.g., an in-line reading. For example, the process may be used on an incoming water supply to test the water quality. The test apparatus can therefore be installed for use in testing an existing incoming water supply and continuously monitor the readings without the need for the withdrawal of samples for benchtop spectroscopic testing. Thus, the spectroscopic detector may be set at the desired wavelength or wavelength range, and then operate effectively in a passive mode, e.g., stare mode, to provide continuous measurements of the fluid supply at the desired detector range as the fluid flows through the spectrophotometer cell.

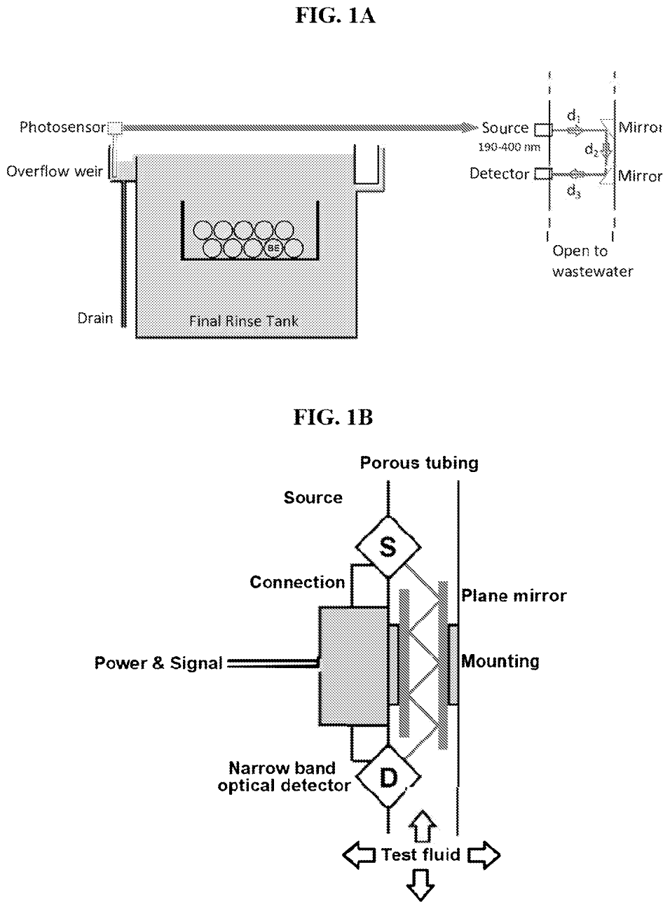

FIG. 1A depicts an exemplary arrangement for using a real-time sensor for assessing the BET content of a source of fluid. The photosensor can be removably placed within the overflow weir of a source of fluid (e.g., a rinse tank) for spectroscopic detection of BET by assessing absorption of light intensity at a wavelength of 190-220 nm. FIG. 1B depicts details concerning the sensor apparatus itself, including the tubing through which a sample volume of fluid continuously flows from a source of test fluid. The tubing represents the spectrophotometer cell that may have a path length of about 8 to about 50 mm. Thus, in some embodiments, the bacterial endotoxin may be spectroscopically sensed on a continuous basis as the fluid sample passes through the spectrophotometer cell pursuant to in-line testing.

In order to enhance the sensitivity of the detection of BET, the present methods may include incorporating a detergent into the fluid sample. It has presently been discovered that the use of one or more specifically selected detergents can enable spectroscopic detection of bacterial endotoxin in amounts as low as 0.003 EU/mL, such as when endotoxin is present in the fluid sample in an amount of about 0.003, 0.004, 0.005, 0.006, 0.007, 0.008, 0.009, or 0.01 EU/mL. Thus, in accordance with the present methods, the fluid sample may further comprise a detergent that does not produce spectral interference in the spectral analytical range of 190-290 nm, below 220 nm, 190-220 nm, or at any particular wavelength as previously described at which spectroscopic absorbance is sensed. The detergent may be present in the fluid sample in a concentration of about 0.01-0.10% by volume. For example, the detergent may be present in the fluid sample in a concentration of about 0.01, 0.02, 0.03, 0.04, 0.05, 0.06, 0.07, 0.08, 0.09, or 0.1% by volume. The detergent may be aliphatic. In some embodiments, the detergent is aliphatic, and lacks one or more of alcoholic, phenolic, or furan functional groups. The detergent may lack each of alcoholic, phenolic, and furan functional groups. An exemplary detergent, the use of which is discussed infra in Example 3, is sodium dodecyl sulfate. In some embodiments, the sodium dodecyl sulfate may be present in the fluid sample in a concentration of about 0.05% by weight. Other detergents can include cetyltrimethylammonium bromide (CTAB), and the general class that includes alpha olefin surfactants, alkane sulfonates, alkyl sulfates (though not likely include primary and secondary alcohol ethoxylates and ethoxysulfates).

Also disclosed herein are kits for detecting bacterial endotoxin in a fluid sample or determining a bacterial source of an endotoxin in a fluid sample comprising a spectrophotometer that is configured to scan the fluid sample at a wavelength in the range of 190-220 nm, a spectrophotometer cell having a radiation path length of about 8 to about 50 mm for containing the fluid sample during scanning by the spectrophotometer, and, a detergent that does not produce spectral interference in the spectral analytical range of 190-220 nm for adding to the fluid sample in a concentration of about 0.01-0.10% by volume. The characteristics of the fluid sample, source of fluid, spectrophotometer, spectrophotometer cell, system configuration (e.g., inline or offline), detection wavelength, limit of detection, detergent type, and detergent concentration may respectively be in accordance with any of the embodiments of these parameters as described above in connection with the presently disclosed methods for detecting bacterial endotoxin in a fluid sample.

The present disclosure also pertains to methods for identifying a bacterial source of an endotoxin in a fluid sample comprising exposing the fluid sample to ultraviolet radiation, spectroscopically sensing the absorbance of the ultraviolet radiation by the sample at a wavelength in the range of 190-220 nm, increasing the concentration of the endotoxin in the fluid sample until an inflection in the absorbance spectrum sensed from the sample is observed, and, correlating the concentration of the endotoxin at which the inflection occurs to the bacterial source of the endotoxin. It has previously been observed that bacterial endotoxin, for example, as exemplified when using Control Standard Endotoxin (CSE) does not follow the Beer-Lambert law relationship, that is, it does not exhibit a linear relationship between the concentration of endotoxin and the observed absorbance values. See Hoi Ting Wong. Polymyxin B-modified Glass Beads: An Alternative Approach to Reduce Lipopolysaccharide Toxicity in Blood. A thesis submitted in conformity with the requirements for the degree of Master of Science Department of Chemistry, University of Toronto (2015). Due to amphipathic properties, endotoxin molecules tend to form multimeric clusters in aqueous solution above a critical micelle concentration (CMC). At concentrations below the onset of such aggregation, the Beer-Lambert law relationship does appear to apply, but following onset of the CMC, the apparent optical density of the sample decreases with increasing concentration, which is spectroscopically observed as an inflection point in the spectral response data. Id.

The present inventors have surprisingly discovered that the onset of the CMC within a fluid sample—as indicated by the inflection in spectral response data—can be used to identify the particular bacterial species that is the origin of the endotoxin present in the sample. By gradually increasing the concentration of endotoxin in a fluid sample and noting the concentration at which the inflection point in the absorbance spectrum occurs, the bacterial source of the endotoxin can be determined. Increasing the concentration of endotoxin may be active or passive. For example, the concentration can be increased by allowing the bacterial population in the fluid sample or source of the fluid sample to grow until the resulting endotoxin concentration that is produced by the population reaches the CMC. Accordingly, increasing the concentration of endotoxin in the sample may be by permitting natural proliferation of the bacterial source of the endotoxin. Another approach for increasing the concentration of endotoxin is by gradually removing fluid that does not contain endotoxin from the sample until the relative concentration of endotoxin in the sample reaches the CMC. A further way to increase the concentration of endotoxin is by addition of additional quantities of the endotoxin to the sample from a further source of the endotoxin. Any approach for increasing the concentration of endotoxin in a fluid sample may be used in accordance with the present methods.

### EXAMPLES

The present invention is further defined in the following Examples. It should be understood that these examples, while indicating preferred embodiments of the invention, are given by way of illustration only, and should not be construed as limiting the appended claims From the above discussion and these examples, one skilled in the art can ascertain the essential characteristics of this invention, and without departing from the spirit and scope thereof, can make various changes and modifications of the invention to adapt it to various usages and conditions.

### Example 1—Determination of Absorbance Wavelength for Detection of Endotoxin

An investigation was conducted for the purpose of assessing the absorbance wavelength for the detection of endotoxin. U.S. Pat. No. 4,195,225 to Karamian (“the Karamian patent”) discloses the use of an analytical range for the assay of endotoxin of 258 nm to 260 nm, and reports a detection sensitivity of 1 ppb to 5 ppb.

Endotoxins are part of the outer cellular membrane of Gram-negative bacteria. Endotoxins are lipopolysaccharides and consist of an O-antigenic region, an outer core and inner core with the latter containing phosphate and amine functionalities and a lipid functional group (lipid-A) containing a species-dependent number of aliphatic chains connected one or more monosaccharides and phosphate moieties. Gram-negative bacteria are commonly associated with water systems and endotoxins pose a risk to patients as they are both pyrogenic and immunogenic. Reducing or eliminating endotoxin from medical devices is necessary to prevent adverse patient outcomes, particularly with devices contacting the brain, spinal cord, cerebrospinal fluid or circulating blood. The U.S. and other pharmacopoeias limit the amounts of endotoxin allowed on medical devices based upon the types of tissue being contacted. Endotoxin quantities are reported in endotoxin units (EU) where one endotoxin unit is equivalent to 0.1 to 0.2 ng of endotoxin.

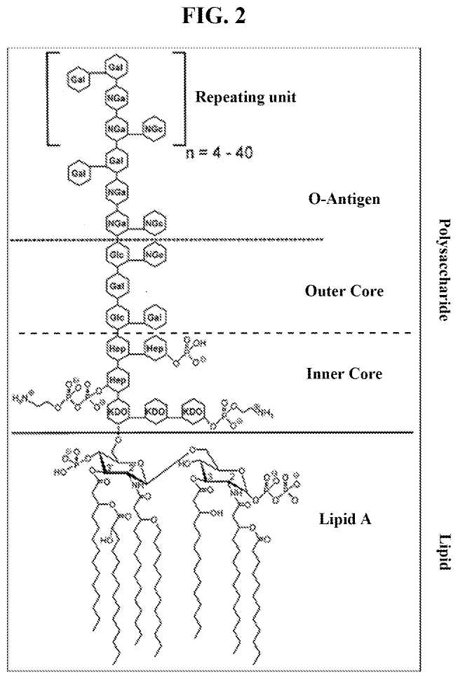

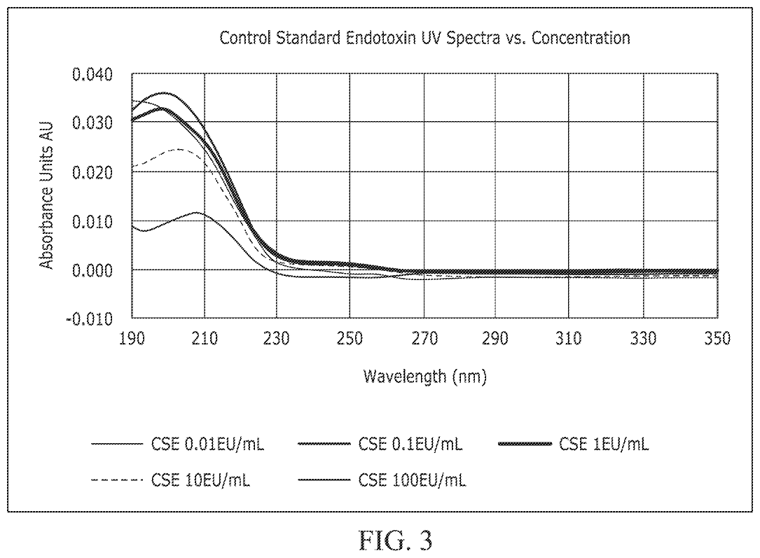

FIG. 2 shows a generalized structure of bacterial endotoxin published by Sigma-Aldrich (“Bacterial Components.” Lipopolysaccharides, Sigma Life Science, Sigma-Aldrich Co., LLC (2011)). Much of this structural information was not known at the time of the Karamian patent or its associated scientific publication, Karamian N A, Waters P F. Spectrophotometry as a tool for assaying endotoxins. J Pharm Sci. 1977 May; 66(5):723-4. As such, the data presented by Karamian warrants interpretation. Karamian, et al. 1977 included a disclosure of UV spectral curves, which indicate strong absorption bands at 258-260 nm for different bacto-lipopolysaccharides, representing bacterial endotoxins. FIG. 3 depicts the spectral response obtained by the present inventors for different concentrations of CSE (used for standardizing limulus amebocyte lysate assays of endotoxin) as detected using a Cary 10 UV-VIS spectrophotometer (Aglient Technologies, Santa Clara, Calif.). As can be seen from FIG. 3, no absorption bands are noted in the 258 nm to 260 nm that was reported by Karamian. In contrast, the experimental data in FIG. 3 includes an absorbance band that is centered approximately at 207 nm.

Interpretation of Karamian data. Based on the now known composition and molecular structure of bacterial endotoxin, there are no chemical moieties in bacterial endotoxin that would give rise to an absorbance at 258 nm to 260 nm. On the other hand, both C═O and N—H functionalities that are presently known to be present within endotoxins could be responsible for the absorbance at approximately 207 nm.

One possible explanation for Karamian's results is contamination of the analyzed sample with nucleic acids. The concentrations of nucleic acids in solution are routinely determined from their strong absorbance at 260 nm. In fact, amounts of nucleic acid are often given as “A260 units” (see, e.g., Franz-Xaver Schmid. Biological Macromolecules: UV-visible Spectrophotometry. ENCYCLOPEDIA OF LIFE SCIENCES/© 2001 Macmillan Publishers Ltd, Nature Publishing Group). If, in the preparation of the endotoxin for analysis, nucleic acids were present as contaminants from the original Gram-negative bacteria, this could explain the spectral responses reported by Karamian. In fact, there is a clear similarity between Karamian's reported absorption bands for endotoxin and the absorption bands for nucleic acids.

Accordingly, although Karamian purported to identify approaches for spectrographic detection of endotoxin, the reported analytical range of 258 nm to 260 nm reflects flawed data.

### Example 2— Spectrophotometric Analysis of Bacterial Endotoxins

Examination of the molecular structure of BET has revealed that there a few functional groups capable of ultraviolet (UV) radiation absorption. Specifically, the following moieties were identified as targets of this investigation prior to experimentation:

CH₃OH(n−σ*) 187 nm, 215 nm

C═O(n−π*) 170 nm-200 nm, 270 nm, 280 nm

H—CH═O (π−π*) 270 nm

The inventors used a Cary 10 UV-VIS spectrophotometer to perform scans using the following instrument and reagent parameters:

Scan Range: 400 nm-190 nm

Resolution: 1 nm

Integration Time: 0.5 seconds

Ordinate: Absorbance (0-1 AU)

Cuvette: 10 mm microcuvette (750 μL); matched pair;

fused quartz

Reference: WFI Quality Water and LRW Water

Samples: CSE @ 0, 0.005, 0.010, 0.015, 0.1, 0.15, 0.2, 1.0, 10 and 100 EU/mL

Detergents: 0.1% aq solutions

Between sample scans, the cuvettes were rinsed thoroughly with deionized water, followed by either WFI Quality Water or LRW, according to the sample matrix and reference solution. A series of CSE (Control Standard Endotoxin CSE 125 μg/vial, Lot #157, ACC Catalog #E0125-1 Exp. 20 May 2021; CSE 0.5 μg/vial, Lot #166, ACC Catalog #E0005-5, Exp. 13 March 2023) samples were prepared for analysis in both of the water types. Using the scan parameters shown above, the data provided in Table 1, below, were obtained.

TABLE 1

| Concentration | | | | | |
|---|---|---|---|---|---|
| EU/mL | A-1 | A-2 | A-3 | Mean A | Std Dev A |
| 0.000 | 0.0000 | 0.0002 | 0.0000 | 0.0001 | 0.0001 |
| 0.005 | 0.0296 | 0.0287 | 0.0290 | 0.0291 | 0.0005 |
| 0.010 | 0.0360 | 0.0360 | 0.0350 | 0.0357 | 0.0006 |
| 0.015 | 0.0284 | 0.0282 | 0.0282 | 0.0283 | 0.0001 |
| 0.100 | 0.0370 | 0.0370 | 0.0370 | 0.0370 | 0.0000 |
| 0.150 | 0.0391 | 0.0397 | 0.0393 | 0.0394 | 0.0003 |
| 0.200 | 0.0059 | 0.0058 | 0.0059 | 0.0059 | 0.0001 |
| 1.000 | 0.0330 | 0.0330 | 0.0330 | 0.0330 | 0.0000 |
| 10.000 | 0.0120 | 0.0120 | 0.0120 | 0.0120 | 0.0000 |
| 100.000 | 0.0250 | 0.0250 | 0.0250 | 0.0250 | 0.0000 |

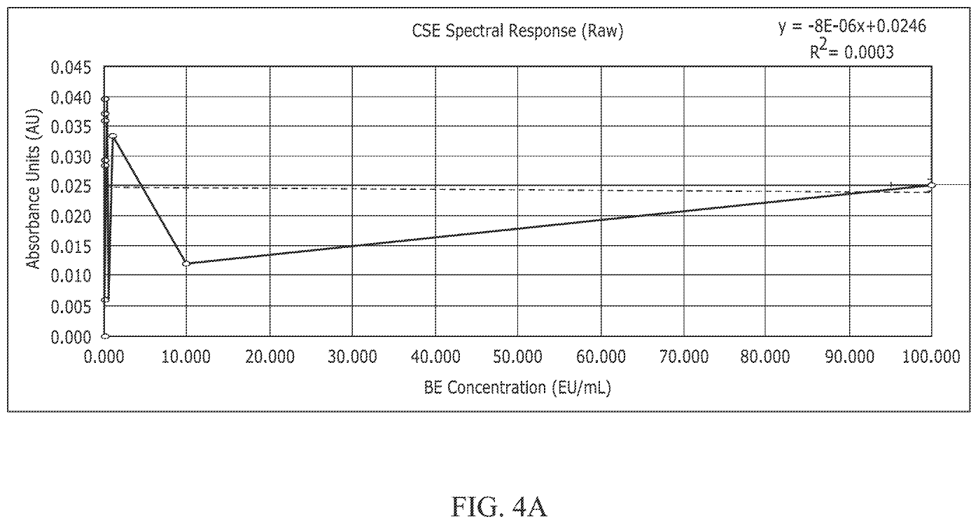

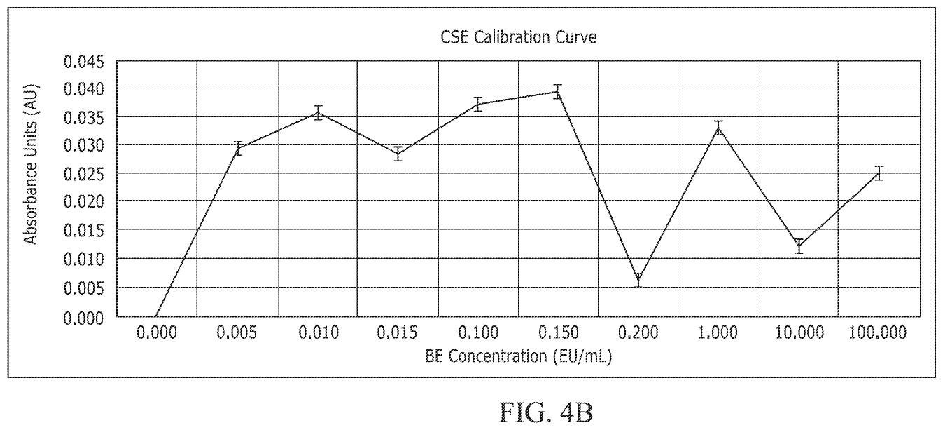

Because of small shifts in the absorbance maxima of the solutions corresponding to concentration, the maximum absorbance is shown rather than using an arbitrary fixed wavelength for this analysis. A review of the results indicates that the response at first glance does not follow the Beer-Lambert Law relationship, that is, it does not exhibit a linear relationship between the concentration of the CSE and the observed absorbance values. This is more easily observed in a graph of the data as shown in FIG. 4A, depicting CSE EU/mL versus absorbance. FIG. 4B provides a logarithmic plot of the data (CSE Log EU/mL versus absorbance).

It is suspected that prior researchers may have found this relationship discouraging and further development was not found in the literature.

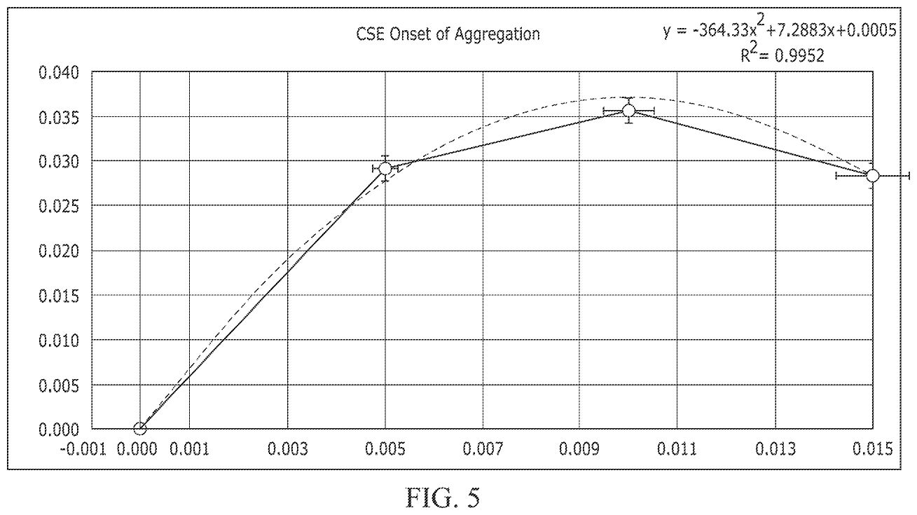

As previously noted, Wong 2015 noted that, due to its amphipathic properties, LPS molecules tend to form multimeric clusters in aqueous solution above a critical micelle concentration (CMC). Spectroscopically, this behavior would be expected to have a profound impact on the correlation of endotoxin concentration and the apparent optical density (absorbance) of the solutions. For example, at concentrations below the onset of aggregation, the mean distance of the LPS molecules in solution would decrease as the concentration was increased—thereby following the Beer-Lambert Law relationship given the presence of a chromophore with sufficient molar absorptivity. However, at the onset of aggregation—what Wong calls the CMC (normally attributed to surface active agents), the mean distance between LPS molecules will begin to increase. This results in an increase of light at the detector of the spectrophotometer as there is less interaction with LPS along the path, τ. As a result, the apparent optical density decreases with increasing concentration. It was predicted by the present inventors that this would result in an inflection of the spectral response data. This was confirmed by examination of the lower concentration samples in the collected data. This is shown FIG. 5, which depicts the inflection of the absorbance data as a result of aggregation. From this result, it was concluded that severe aggregation of the CSE was occurring at approximately 0.01 EU/mL. This is consistent with the observations of Wong and explains the departure from linearity when using this method.

As the concentration is increased beyond this inflection point, larger aggregates form and the spacing between them continues to increase until the aggregates become so large that additional LPS added to the solution begins to decrease the spacing between the aggregates. At this point, as the passage of light through the sample begins to interact with more LPS and less direct light reaches the detector; a quasi-Beer-Lambert Law relationship is reestablished. Refer to FIG. 4A for concentrations between 10 EU/mL and 100 EU/mL to see this effect.

### Example 3—Detergent Selection and Evaluation of Effect of Detergent Use

The inventors considered the use of surface active agents for use during the detection of bacterial endotoxins by ultraviolet spectrophotometry. Three detergents respectively representing different classes of surfactant were selected for study: Tween 80, Triton X-100, and sodium dodecyl sulfate (SDS).

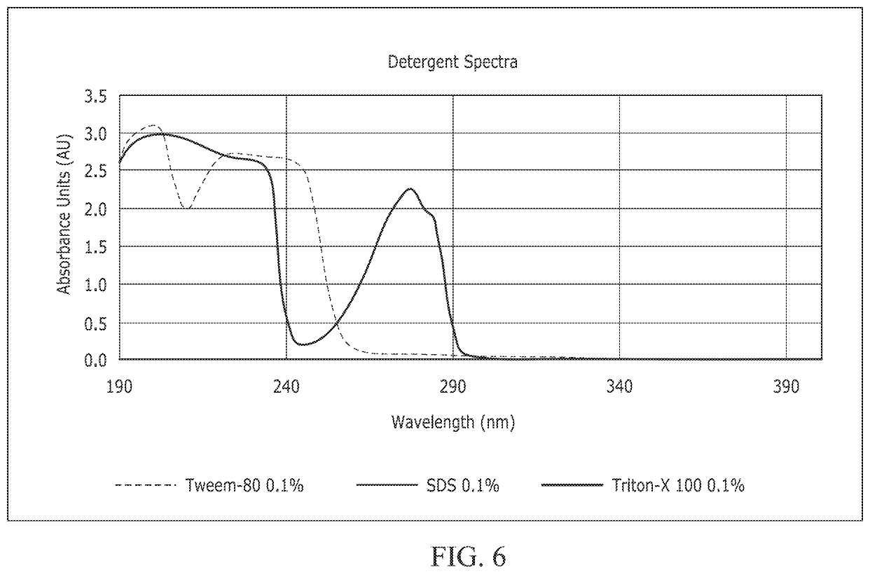

Bacterial endotoxin has been shown experimentally to absorb at 190-220 nm (Example 1, supra). It was therefore important that any detergent selected for use in methods of detecting endotoxin does not spectroscopically interfere at relevant wavelengths. Spectra of 0.1% v/v aqueous solutions of the three detergents (in WFI Quality Water) are shown in FIG. 6. The detergent solutions were scanned using the same instrument parameters as described in Example 2. As can be seen in FIG. 6, both Tween 80 and Triton-X 100 exhibited interfering absorbance characteristics in the spectral region used for endotoxin analysis. In contrast, SDS did not show any appreciable absorbance in the analytical spectral range.

Both Tween 80 and Triton X-100 contain aromatic groups—furan and phenol, respectively. Tween 80 also contains a carbon-carbon double bond and a carbonyl functional group. It was concluded that these functionalities were responsible for the absorption properties of the detergents within the range of 190 nm to 290 nm. SDS is a linear hydrocarbon with a terminal hydrophilic sulfate group and lacks aromaticity or double bonded carbon. As such, it is relatively transparent in the 190 nm to 290 nm spectral range.

For the next portion of the experiment, equal volumes of 0.1% SDS (aq) were mixed with CSE samples and vortex for mixing. This produced solutions containing 0.05% SDS and half the original concentration of CSE. The samples were scanned using the instrument parameters described in Example 2. The results were evaluated in order to address the following questions: 1) does SDS prevent aggregation of BET?; and, 2) does SDS affect the value of c in the Beer-Lambert response?

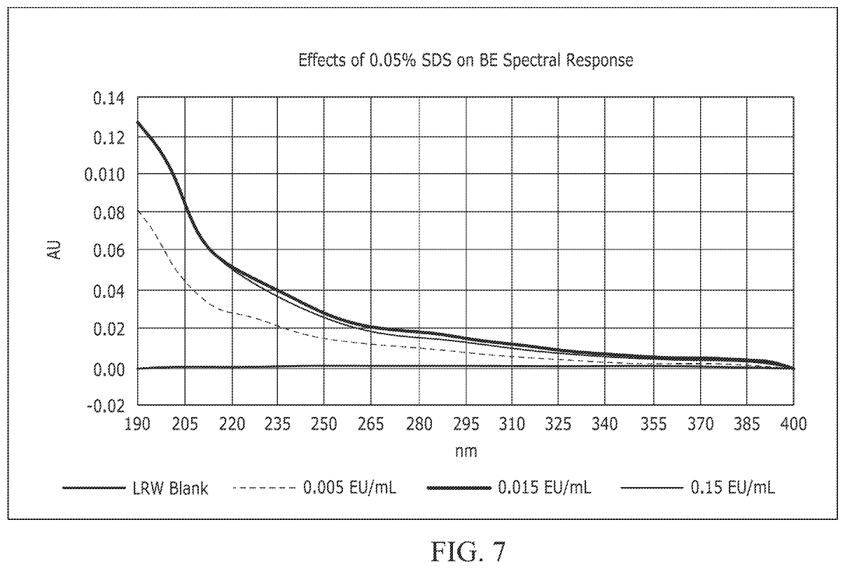

To determine the answer to the first question, four samples were compared for their response—a blank and three CSE samples to which SDS was added. These were then compared to the CSE samples previously scanned that did not contain SDS. The spectral results are shown in FIG. 7. From these results, it is apparent that the responses for the

0.015 EU/mL sample and 0.15 EU/mL sample are not correlated to the absorbance values observed. From this, it can be concluded that the addition of SDS does not affect the agglomeration of endotoxin in water.

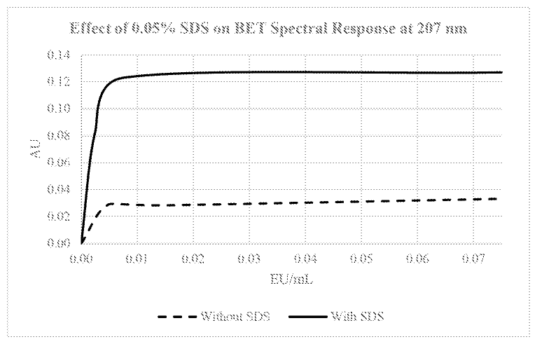

Upon further examination of these results, it was noted that the absorptivity of the CSE was increased substantially by the addition of SDS to the solution. This is demonstrated in the spectra shown in FIG. 8. The presence of SDS enhanced the absorption results from 285% to 435% in this experiment. This increased sensitivity of the method therefore lowers the limit of detection (LOD) of the inventive methods to 0.003 EU/mL. By comparison, the gel clot method has an LOD of 0.01 to 0.03 EU/mL, and the chromogenic method LOD is 0.005 EU/mL. This makes the presently disclosed spectrophotometric methods comparable in the level of detection of endotoxin, barring the presence of interfering substances. Without wishing to be bound by any particular theory of operation, the enhancement effect from the use of detergent may reflect a change in the molar extinction coefficient of endotoxin by its presence in the aqueous environment.

In view of the above-described results, it was concluded that when selecting detergent to use with ultraviolet spectral analysis of bacterial endotoxins, the molecular structure should not contain aromatic groups or carbon-carbon double bonds in order to avoid interference in the spectral analytical range of 190 nm to 290 nm. Primary and secondary alcohol functionality is also expected to interfere with absorption in to 200 nm to 210 nm range. It is believed that classes of detergents suitable for use include those containing only singly-bonded carbon (aliphatic) detergents lacking alcoholic, phenolic, and furan functionalities. It was also concluded that the presence of a detergent can influence the molar absorptivity of the endotoxin and can provide a significantly enhanced signal in the analytical procedure.

What is claimed:

1. A method for detecting bacterial endotoxin in a fluid sample comprising:
exposing the fluid sample to ultraviolet radiation;
spectroscopically sensing absorbance of the ultraviolet radiation by the sample at a wavelength in the range of 190-220 nm; and,
based on the sensed absorption, determining whether the bacterial endotoxin is absent or present in the sample.

2. The method according to claim 1, wherein the absorbance of the ultraviolet radiation is sensed at a wavelength in the range of 205-210 nm.

3. The method according to claim 1, wherein the absorbance of the ultraviolet radiation is sensed at a wavelength of 207 nm.

4. The method according to claim 1, wherein the bacterial endotoxin is detected when present in the fluid sample in an amount of about 0.01 EU/mL to about 10 EU/mL.

5. The method according to claim 1, wherein the bacterial endotoxin is detected when present in the fluid sample in an amount of about 0.1 EU/mL to about 10 EU/mL.

6. The method according to claim 1, wherein the bacterial endotoxin is spectroscopically sensed while the sample is contained within a spectrophotometer cell having a radiation path length of about 8 to about 50 mm.

7. The method according to claim 6, wherein the bacterial endotoxin is spectroscopically sensed on a continuous basis as the sample passes through the spectrophotometer cell pursuant to in-line testing.

8. The method according to claim 1, wherein the fluid sample further comprises a detergent that does not produce spectral interference in the spectral analytical range of 190-220 nm.

9. The method according to claim 8, wherein the detergent is aliphatic.

10. The method according to claim 8, wherein the detergent lacks alcoholic, phenolic, or furan functional groups.

11. The method according to claim 8, wherein the detergent is present in the fluid sample in a concentration of about 0.01-0.10% by volume.

12. The method according to claim 8, wherein the detergent is sodium dodecyl sulfate.

13. The method according to claim 12, wherein the detergent is present in the fluid sample in a concentration of about 0.05% by volume.

14. The method according to claim 8, wherein the bacterial endotoxin is detected in an amount of as low as 0.003 EU/mL.

15. A method for identifying a bacterial source of an endotoxin in a fluid sample comprising:
exposing the fluid sample to ultraviolet radiation;
spectroscopically sensing the absorbance of the ultraviolet radiation by the sample at a wavelength in the range of 190-220 nm;
increasing the concentration of the endotoxin in the fluid sample until an inflection in the absorbance spectrum sensed from the sample is observed; and,
correlating the concentration of the endotoxin at which the inflection occurs to the bacterial source of the endotoxin.

16. The method according to claim 15, wherein the absorbance of the ultraviolet radiation is sensed at a wavelength in the range of 205-210 nm.

17. The method according to claim 15, wherein the absorbance of the ultraviolet radiation is sensed at a wavelength of 207 nm.

18. The method according to claim 15, wherein the bacterial endotoxin is spectroscopically sensed while the sample is housed in a spectrophotometer cell having a radiation path length of about 8 to about 50 mm.

19. The method according to claim 15, wherein the concentration of the endotoxin in the sample is as low as 0.003 EU/mL prior to increasing the concentration of the endotoxin.

20. The method according to claim 15, wherein increasing the concentration of the endotoxin in the fluid sample is by natural proliferation of the bacterial source of the endotoxin.

21. The method according to claim 15, wherein increasing the concentration of the endotoxin in the fluid sample is by addition of the endotoxin to the sample from a further source of the endotoxin.

22. A kit for detecting bacterial endotoxin in a fluid sample or determining a bacterial source of an endotoxin in a fluid sample comprising:
a spectrophotometer that is configured to scan the fluid sample at a wavelength in the range of 190-220 nm;
a spectrophotometer cell having a radiation path length of about 8 to about 50 mm for containing the fluid sample during scanning by the spectrophotometer; and,
a detergent that does not produce spectral interference in the spectral analytical range of 190-220 nm for adding to the fluid sample in a concentration of about 0.01-0.10% by volume.
